(19) United States
(12) Patent Application Publication
(10) Pub. No.: US 2004/0073246 A1
(43) Pub. Date: Apr. 15, 2004

(54) Cutter head for a microkeratome

(75) Inventors: Jean-Luc Aufaure, Souvigny (FR); David Frileux, Marigny (FR)

Correspondence Address: YOUNG & THOMPSON, 745 SOUTH 23RD STREET 2ND FLOOR, ARLINGTON, VA, 22202

(73) Assignee: MORIA SA, ANTONY (FR)

(21) Appl. No.: 10/676,471

(22) Filed: Oct. 2, 2003

(30) Foreign Application Priority Data
Oct. 8, 2002 (FR) 02 12457

(51) Int. Cl. A61F 9/00

(52) U.S. Cl. 606/166

(57) ABSTRACT

A microkeratome cutter head comprising a body and a cutter blade slidably mounted in a housing passing through the body in a direction substantially parallel to the cutting edge of the blade, wherein the blade possesses a slot parallel to its cutting edge and opening out into one of the side edges of the blade, while the housing possesses a partition received in the slot of the blade as it is inserted into the body, and co-operating with the edges of the slot to guide the blade in its motion parallel to its cutting edge.

### BACKGROUND OF THE INVENTION

A microkeratome is an ophthalmological surgical instrument used for performing resection of the cornea, and in particular for cutting free a flap of the cornea so as to perform corrective ablation in the uncovered stroma. The instrument comprises a ring for fixing on the eye on which surgery is to be performed, the ring forming a circular or rectilinear guide for a cutter head. The cutter head possesses a body provided with guide means corresponding to those of the ring, within which a cutter blade sloping towards the front and towards the ring is driven with reciprocating motion along its cutting edge. In general, in front of the blade, the body possesses a plate for flattening the cornea so that the cutting edge of the blade projects beneath the plate, being spaced apart from the plate by a distance determined as a function of the thickness of the flap to be made in the cornea.

In general, this distance is not adjustable. It is determined by the relative position of the blade in the body of the cutter head. Unfortunately, for numerous reasons, in known instruments the position of the blade is poorly controlled.

In most prior art keratomes, the cutter head includes a transverse housing in which the blade is guided during its reciprocating motion. This housing has a section in a plane perpendicular to the cutting edge which is substantially the same as the section of the blade, ignoring operating clearances. The term “blade” is used to cover an assembly formed by a metal plate having a front edge constituting the cutting edge, like a razor blade, and by an element made of plastics material in the form of a knob fitted to the blade so as to project from one of its faces and referred to as the “shuttle”. This plastics shuttle presents at least one face which forms the sliding support face for the blade and that bears against a corresponding guide surface in the housing of the body. In addition, a groove is formed in this plastics shuttle in which there slides an eccentric finger of the motor that drives the reciprocating motion of the blade when the body of the cutter head is coupled to the casing of the motor.

The shuttle is fitted to the metal plate by overmolding, clip fastening, or adhesive, all of which techniques, regardless of how carefully they are implemented, lead to uncertainty in the accuracy of the relative position between the shuttle and the metal plate proper of the blade. As a result, the distance from the sliding face of the shuttle in the housing of the head body from the cutting edge can vary from one blade to another, and for any one blade it is not known with sufficient accuracy to ensure that the position of the cutting edge relative to the flattening plate is properly controlled in operation. Together with other parameters, this lack of accuracy has a significant influence on the thickness of the corneal flap. However the present trend in this field is towards making corneal flaps of ever decreasing thickness.

### OBJECT OF THE INVENTION

The present invention seeks to remedy this drawback, at least in part.

### BRIEF DESCRIPTION OF THE INVENTION

The present invention provides a microkeratome cutter head comprising a body and a cutter blade slidably mounted in a housing passing through the body in a direction substantially parallel to the cutting edge of the blade, wherein the blade possesses a slot parallel to its cutting edge and opening out into one of the side edges of the blade, while the housing possesses a partition received in the slot of the blade as it is inserted into the body, and co-operating with the edges of the slot to guide the blade in its motion parallel to its cutting edge.

This provide direct guidance between the head and the blade enabling uncertainties in the assembly between the blade and its drive shuttle to be overcome, which shuttle was used in the prior art to guide the blade in the housing of the head.

In a preferred embodiment, the partition is shorter in length than the housing in the sliding direction, thus defining an inside end of the housing which forms an abutment for positioning the blade in the housing. This characteristic serves firstly, while the blade is being inserted into the head, to prevent the blade escaping from the person handling it and coming out through the other side of the through housing, and secondly it forms an abutment for putting the blade in a determined position transversely inside the housing of the head, thus making it easier to couple the blade quickly with its drive means inside the body of the head.

In other embodiments, the partition may be constituted by no more than one or two pegs perpendicular to the plane of the blade housing in which the blade reciprocates, the pegs possibly being metal pegs in a metal or plastics head, and being fitted in any conventional manner.

Specifically for the purpose of enabling it to be driven by conventional means, the blade has an oblong opening substantially perpendicular to the slot for receiving a drive finger rotating eccentrically about an axis orthogonal to the slot. In addition, the housing possesses a recess beneath the blade in register with said oblong opening, the recess making it possible for the drive finger that passes through the blade to rotate.

In a variant embodiment, provision is made in conventional manner to fit a drive knob on one of the faces of the blade to enable the blade to co-operate with the drive finger. Under such circumstances, the portion of the housing passing through the body in which the knob of the blade slides is of a profile that is sufficiently large to ensure that there is no contact between the body and the knob.

Other characteristics and advantages appear from the description given below by way of indication, and relating to an embodiment of the invention and to variants thereof.

### BRIEF DESCRIPTION OF THE DRAWINGS

Reference is made to the accompanying drawings, in which:

FIG. 1 is an outside view of a pivoting keratome head in accordance with the invention;

FIG. 2 is a section view of said head on plane P of FIG. 1, inside the housing passing through the head;

FIG. 3 shows a first variant of the blade of the invention;

FIG. 4 is a diagrammatic side view of a variant of the cutter head of the invention;

FIGS. 5 and 6 are a front view and a section view of a blade adapted to the FIG. 4 head; and

FIG. 7 is a diagrammatic section view of a variant embodiment of the invention.

### DETAILED DESCRIPTION OF THE INVENTION

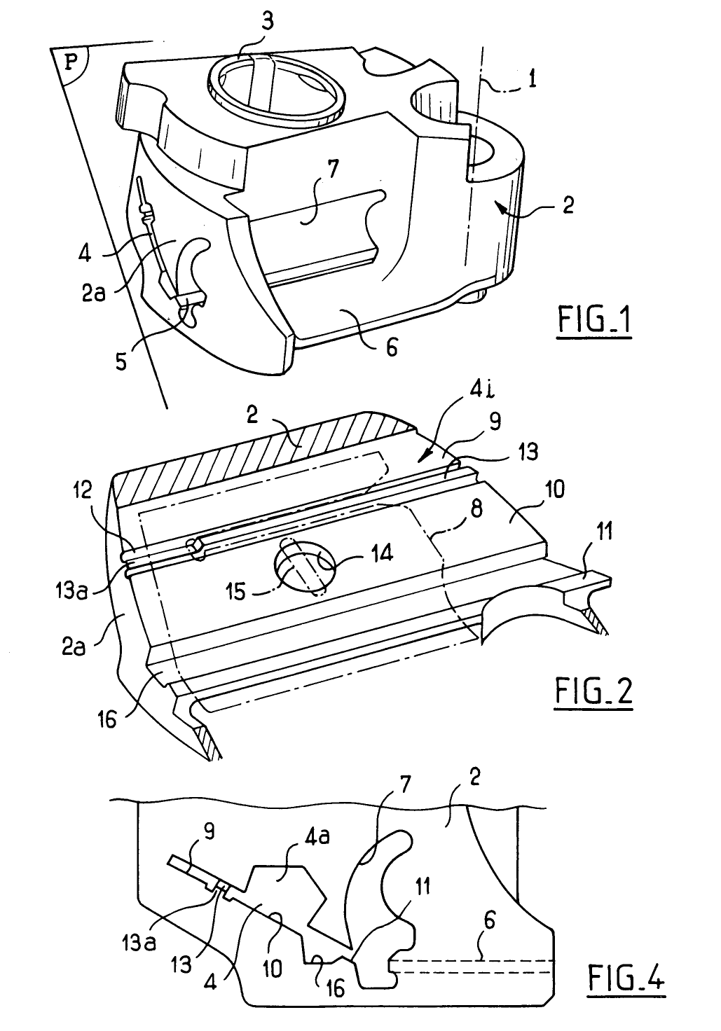

The head shown in FIG. 1 is a head of known shape for fitting to a motor-driven microkeratome pivoting about a vertical axis 1 carried by the ring for fixing to the eye. The body 2 of the head may be connected at 3 to a motor unit which serves firstly to rotate the head about the axis 1 and secondly to impart reciprocating motion to a blade placed in a housing 4 of the body that opens out at 5 close to the rear edge of a flattening plate 6. The flap of the cornea that is cut free by the blade oscillating in the head slides over a curved deflector 7.

The dispositions of the invention can be seen more clearly in FIG. 2 which is a section view of the FIG. 1 head on a plane P occupying the housing 4 that passes through the body 2 of the head of the microkeratome. FIG. 2 thus shows the bottom surface 4i of the housing 4 on which the blade 8 rests (the blade being represented by a chain-dotted line). This bottom surface possesses three blade support zones referenced 9, 10, and 11 going from the rear of the head to the end of the housing close to the flattening plate 6. These surfaces 9, 10, and 11 lie in a common sloping plane. The surface 9 is separated from the surface 10 by a groove 12 having a partition 13 standing on the bottom thereof and projecting proud of the plane common to the surfaces 9, 10, and 11, and optionally, where appropriate, meeting the top surface of the housing 4. This partition 13 does not extend over the full length of the housing 4; at the end 2a of the body of the cutter head through which the blade is inserted into the housing, the partition possesses a zone 13a of reduced height extending to a level below the plane of the surfaces 9, 10, and 11. The surface 10 is also provided with a recess 14 which is situated beneath the blade 8 and leaves room for the eccentric finger to move so as to impart reciprocating motion to the blade, said finger forming part of the motor unit connected to the cutter head at 3. The eccentric finger (not shown) passes through the blade 8 via an oblong opening 15 which extends perpendicularly to the cutting edge F. Finally, a channel 16 lies between the middle surface 10 and the last surface 11 of the housing 4 and constitutes a receptacle for any particles that might be produced during motion of the blade 8 in the head 2.

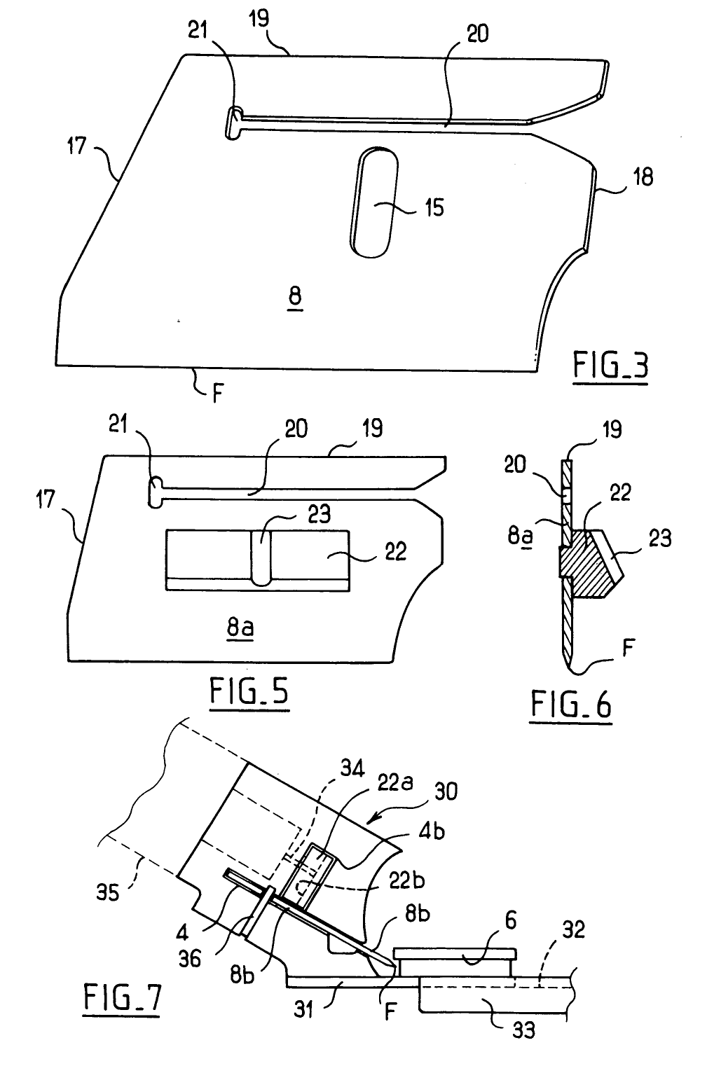

The blade 8 is shown on its own in FIG. 3. It is constituted by a thin metal plate having a cutting edge F, two side edges 17 and 18 and a rear edge 19. On top, close to the rear edge 19, it includes a slot 20 extending parallel to the cutting edge F and opening out into the side edge 18, with its opposite end being formed by a recess 21 provided for reasons associated with machining and preparing the slot 20. The slot 20 flares towards the edge 18, with this flare making it easier to insert the blade 8 into the housing 4 through the end 2a of the body 2 when the slot 20 takes up a position astride the partition 13 beyond its zone of reduced height 13a. When the blade is pushed fully into the housing 4, the partition 13 comes into abutment against the end of the recess 21 so that the oblong opening 15 in the blade lies over the recess 14 at a location intersecting the circular path of the drive finger of the motor unit when the motor unit is connected to the cutter head 2. In conventional manner, the drive finger is resiliently retractable along its own axis so that when the head is mounted on the motor unit, the finger can retract on coming into contact with the top surface of the blade 8, and then on first rotation it can penetrate into the oblong opening 15.

FIG. 4 shows a variant embodiment of the cutter head, seen in profile, where the blade housing 4 is surmounted by a recess 4a for accommodating the knob or shuttle 22 of a variant embodiment 8a of the blade as shown in FIGS. 5 and 6. Elements that are described above and that also appear in these FIGS. 4, 5, and 6 are given the same references. It should be observed that in conventional manner it is a notch 23 of the shuttle 22 which receives the eccentric finger for driving the blade. The profile of the portion 4a of the housing receiving the shuttle 22 is higher than the profile of the shuttle so as to ensure that no contact can occur between the shuttle and the body of the cutter head.

The slot 20 in a blade 8 or 8a can be made in extremely accurate manner relative to the cutting edge. Similarly, it is also possible to make the partition 13 at a well-controlled distance from the flattening plate 6, such that when the blade is inserted the cutting edge is accurately positioned relative to the flattening plate 6. This eliminates the positioning uncertainty that used to exist due to the blade being guided in the cutter head by means of the shuttle. This ensures that the thickness of the corneal flap is better controlled.

Co-operation between the slot 20 and the partition 13 also performs a keying function to ensure that the blade is properly inserted in the head.

The description above is given in the context of a pivoting cutter head, however all of the dispositions of the invention are equally applicable to a cutter head that advances in rectilinear manner perpendicularly to the cutting edge of the blade over the ring for fixing to the eye.

Although the guidance for the blade in the cutter head is described above as being provided by co-operation between the slot 20 and a partition 13, i.e. a rectilinear projection extending over a certain length, it would not go beyond the ambit of the invention to replace the partition with one or two pegs with the slot 20 of the blade 8, 8a coming astride the peg(s). FIG. 7 is a diagram of this variant embodiment in a head that advances in rectilinear manner. The base of the body 30 of the cutter head carries two male dovetails 31 capable of sliding in female dovetails 32 formed in an eye-fixing ring 33. The blade 8b possesses a shuttle 22a housed in a recess 4b of the housing 4 that does not come into contact with the shuttle or knob 22a. The shuttle 22a is substantially perpendicular to the blade 8b and co-operates via a notch 22b with a drive finger such as 34 belonging to a motor unit 35. A peg 36 (or preferably two spaced-apart pegs 36) pass through the housing 4 for the blade 8b in the body 30 and the slot 20 is placed astride them. Like the partition 13, they constitute means for guiding the blade in the head during its transverse reciprocating motion.

What is claimed is:

1. A microkeratome cutter head comprising a body and a cutter blade slidably mounted in a housing passing through the body in a direction substantially parallel to the cutting edge of the blade, wherein the blade possesses a slot parallel to its cutting edge and opening out into one of the side edges of the blade, while the housing possesses a projection received in the slot of the blade as it is inserted into the body, and co-operating with the edges of the slot to guide the blade in its motion parallel to its cutting edge.

2. A cutter head according to claim 1, wherein the projection is a partition of length shorter than the length of the housing in the sliding direction, thereby defining an inside end of the housing that forms an abutment for positioning the blade in the housing.

3. A cutter head according to claim 1, wherein the projection is constituted by at least one peg extending perpendicularly to the plane of the cutter blade.

4. A cutter head according to claim 2, wherein the blade includes an oblong opening substantially perpendicular to the slot for receiving a drive finger turning eccentrically about an axis orthogonal to the slot.

5. A cutter head according to claim 4, wherein the housing possesses a recess beneath the blade in register with the oblong slot allowing the drive finger to rotate.

6. A cutter head according to claim 2, wherein the blade is fitted on one of its faces with a drive knob provided with a notch extending orthogonally to the slot for receiving a drive finger rotating eccentrically about an axis orthogonal to the slot.

7. A cutter head according to claim 6, wherein the profile of the portion of the housing through the body in which the knob of the blade slides does not come into contact with the knob.

8. A cutter head according to claim 1, wherein the housing possesses at least one channel parallel to the partition and situated between the partition and the cutting edge of the blade, forming a cavity for collecting particles.

9. A microkeratome cutter blade comprising a metal plate with a cutting edge and two side edges connecting the ends of the cutting edge to a rear edge, the blade including a slot parallel to the cutting edge and opening out into one of its side edges.

10. A blade according to claim 9, wherein the slot opens out into the side edge in flared manner.

11. A blade according to claim 9, including an oblong opening perpendicular to the cutting edge.

12. A blade according to claim 9, including a drive knob projecting from one of its faces.
